(19) United States
(12) Patent Application Publication
(10) Pub. No.: US 2008/0140202 A1
(43) Pub. Date: Jun. 12, 2008

(54) Energy-Storing Spinal Implants and Methods of Use

(76) Inventors: Randall Noel Allard, Germantown, TN (US); Larry Thomas McBride, Memphis, TN (US); Tom J. Francis, Cordova, TN (US); Thomas A. Carls, Memphis, TN (US)

Correspondence Address: COATS & BENNETT/MEDTRONICS, 1400 CRESCENT GREEN, SUITE 300, CARY, NC, 27518, US

(21) Appl. No.: 11/608,363

(22) Filed: Dec. 8, 2006

(51) Int. Cl. A61F 2/44; A61B 17/08

(52) U.S. Cl. 623/17.13; 623/17.11; 606/151

(57) ABSTRACT

An implant for insertion between vertebral bodies in a patient includes a flexible tether that is coupled between fasteners attached to the vertebral bodies. An energy storing device may be coupled to the tether to convert extension forces between the vertebral bodies into potential energy. The energy storing device may reduce defects at the interface between the fasteners and the vertebral bodies. Various embodiments are provided, including energy storing devices implemented as springs, leaf springs, coiled wire, and corrugated shapes. The energy storing device may be preloaded between the vertebral bodies to further reduce shock from sudden extensions. The energy storing device may be secured to the tether before or after the tether is secured to the fasteners. Further, the energy storing device may be sized to allow the tether and energy storing device to pass laterally through a fastener opening.

### BACKGROUND

Spinal implants are often used in the surgical treatment of spinal disorders such as degenerative disc disease, tumors, disc herniations, scoliosis or other curvature abnormalities, and fractures. Many different types of treatments are used, including the use of dynamic implants to preserve motion between vertebral members. One particular treatment contemplates one or more flexible tethers that are secured to vertebral members to constrain growth or over-distraction in a particular direction while permitting compression and relative motion between vertebral members. This treatment may offer the advantage of controlling growth and healing of the spine without fusing one or more vertebral levels.

The tethers that are used in this particular treatment are flexible. Further, some tethers may include an inelastic structure that does not appreciably stretch in the longitudinal direction. Other tethers may include an elastic structure that stretches a nominal amount in the longitudinal direction under the influence of a distraction force. In either case, the tethers may be anchored to the vertebral members using conventionally known hardware such as screws, plates, or staples. A problem with conventional solutions is that the tethers have a limited extension range. Once the limit of tether extension is reached, additional distraction of the vertebral members tends to stress the interface between the securing hardware and the vertebral member. In extreme cases, the hardware may tend to plow or otherwise extract from the vertebral member to which the hardware is anchored. Therefore, while conventional tether solutions may provide desired flexibility, they may not provide enough extension range or other buffer to prevent damage to the anchor points at which the tethers are secured.

### SUMMARY

Illustrative embodiments disclosed herein are directed to an implant for insertion between vertebral bodies in a patient that includes a flexible tether that is coupled between fasteners attached to the vertebral bodies. An energy storing device may be coupled to the tether to convert extension forces between the vertebral bodies into potential energy. The energy storing device may reduce defects at the interface between the fasteners and the vertebral bodies. Various embodiments are provided, including energy storing devices implemented as springs, leaf springs, coiled wire, and corrugated shapes. Each of the various embodiments may include one or more apertures so that the tether may be threaded through the energy storing device. The energy storing device may be preloaded between the vertebral bodies to further reduce shock from sudden extensions. In one embodiment, the energy storing device may include an increasing moment arm to stabilize the extension force required to deflect the energy storing device. The energy storing device may be secured to the tether before or after the tether is secured to the fasteners. Further, the energy storing device may be sized to allow the tether and energy storing device to pass laterally through a fastener opening.

### BRIEF DESCRIPTION OF THE DRAWINGS

FIG. 1 is a side elevation view of a vertebral implant according to one embodiment secured to multiple vertebral members;

FIG. 2 is a side view of a vertebral implant secured to multiple vertebral members and including a linear energy storing device;

FIG. 3 is a side view of a vertebral implant secured to multiple vertebral members and including a varying energy storing device;

FIG. 4 is a perspective view of an energy storing device according to one embodiment;

FIG. 5 is a side view of an energy storing device attached to vertebral tether in a neutral state according to one embodiment;

FIG. 6 is a side view of an energy storing device attached to vertebral tether in an extended state according to one embodiment;

FIG. 7 is a graphical representation of an exemplary force-displacement relationship for an energy storing device according to one embodiment;

FIG. 8A is a perspective view of an energy storing device according to one embodiment;

FIG. 8B is a perspective view of an energy storing device according to one embodiment;

FIG. 9A is a side view of an energy storing device attached to vertebral tether in a neutral state according to one embodiment;

FIG. 9B is a side view of an energy storing device attached to vertebral tether in an extended state according to one embodiment;

FIG. 10 is a frontal view of an energy storing device in a neutral state according to one embodiment;

FIG. 11A is a side view of an energy storing device in a neutral state according to one embodiment;

FIG. 11B is a side view of an energy storing device in an extended state according to one embodiment;

FIG. 12 is a frontal view of an energy storing device in a neutral state according to one embodiment;

FIG. 13A is a side view of an energy storing device in a neutral state according to one embodiment;

FIG. 13B is a side view of an energy storing device in an extended state according to one embodiment;

FIG. 14A illustrates exemplary tether routing through an energy storing device in a neutral state according to one embodiment;

FIG. 14B illustrates exemplary tether routing through an energy storing device in an extended state according to one embodiment;

FIG. 15A illustrates exemplary tether routing through an energy storing device in a neutral state according to one embodiment;

FIG. 15B illustrates exemplary tether routing through an energy storing device in an extended state according to one embodiment;

FIG. 16A is a side view of an energy storing device in a neutral state according to one embodiment;

FIG. 16B is a side view of an energy storing device in an extended state according to one embodiment;

FIG. 17A is a side view of an energy storing device in a neutral state according to one embodiment;

FIG. 17B is a side view of an energy storing device in an extended state according to one embodiment;

FIG. 18A is a side view of an energy storing device in a neutral state according to one embodiment;

FIG. 18B is a side view of an energy storing device in an extended state according to one embodiment;

FIG. 19 is a perspective view of an energy storing device according to one embodiment;

FIG. 20A is a side view of an energy storing device in a neutral state according to one embodiment;

FIG. 20B is a side view of an energy storing device in an extended state according to one embodiment;

FIG. 21 is a perspective view of an energy storing device according to one embodiment;

FIG. 22 is a side view of an energy storing device exhibiting an increasing moment arm during extension according to one embodiment;

FIG. 23 is a graphical representation of an exemplary force-displacement relationship for an energy storing device according to one embodiment;

FIG. 24 is a side view of an energy storing device exhibiting an increasing moment arm during extension according to one embodiment;

FIG. 25 is a side view of an energy storing device exhibiting an increasing moment arm during extension according to one embodiment;

FIGS. 26A-B depict an exemplary process for securing an implant to multiple vertebral members according to one embodiment;

FIGS. 27A-B depict an exemplary process for securing an implant to multiple vertebral members according to one embodiment; and

FIGS. 28A-C depict an exemplary process for securing an implant to multiple vertebral members according to one embodiment.

### DETAILED DESCRIPTION

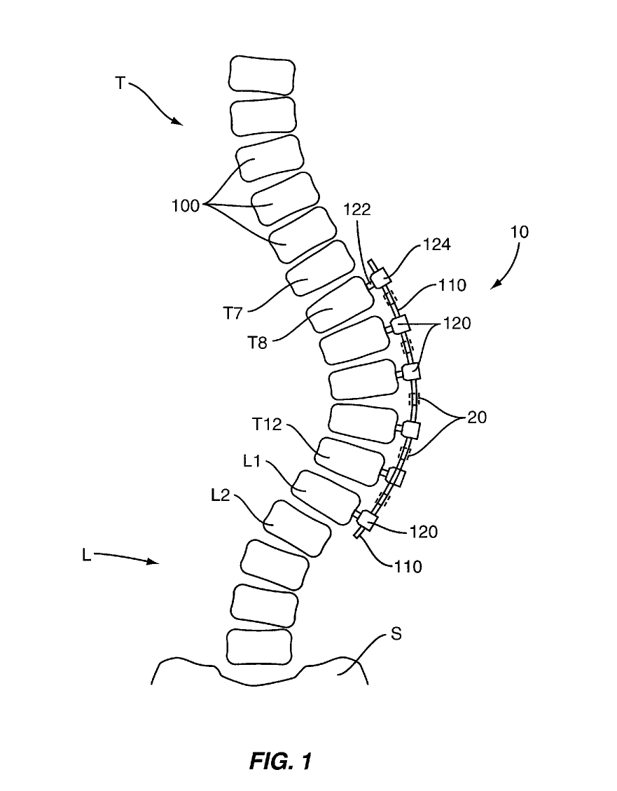

The various embodiments disclosed herein are directed to energy-storing devices that are used in conjunction with flexible tethers that are secured to vertebral members. The devices may form a part of the tether or may be a separate member that is secured to the tether. The devices maintain the flexible characteristic of the tethers, but advantageously increase the range of tether extension to reduce the risk of anchor defects. The devices are further characterized as energy-storing devices in that distraction forces are stored as elastic energy to reduce stress on the tether anchors. An exemplary implant 10 for supporting vertebral members is illustrated in FIG. 1. FIG. 1 illustrates a patient's spine that includes the vertebral members 100 of the thoracic region T, the lumbar region L, and the sacrum S. In the illustrated application, the spine has a scoliotic curve with an apex of the curve being offset from its correct alignment in the coronal plane. The spine is deformed laterally so that the axes of the vertebral members 100 are displaced from the sagittal plane passing through a centerline of the patient. The implant 10 includes a flexible tether 110 that may be fabricated from a variety of biocompatible materials. For example, the tether 110 may be constructed as fabric, mesh, cable, string, or rod comprising synthetic or natural fibers, polymeric materials, ceramics, or metals. The implant 10 is attached to vertebral members 100 with one or more fasteners 120. That is, the fasteners 120 anchor the tether 110 to each vertebral member 100. As used herein, vertebral member 100 refers to a vertebra including the body, arches, and processes. In the illustrated embodiment, the fasteners 120 comprise bone screws. The fasteners 120 include a threaded shank 122 that is screwed into the vertebral members 100 and a head 124 that is sized to hold and retain the tether 110. The fasteners 120 may be substantially rigid or may include a head 124 that pivots relative to the shank 122 about one or more axes. In other embodiments, the tether 110 may be anchored to the vertebral members 100 with other types of fasteners 120, including but not limited to plates, staples, hooks, cables, or stakes. In other embodiments, the tether 110 may be anchored to other regions of the vertebral members 110, such as the pedicles, laminae, posterior arches, transverse processes, or spinous processes.

Because the tether 110 is flexible, the implant 10 allows flexion, extension, axial rotation, and lateral bending. Meanwhile, because the tether 110 is fixedly attached to the fasteners 120, the implant 10 limits growth and/or distraction about the convex side of the scoliotic curve. These constraints on motion maintain kyphosis, lordosis, and coronal balance while controlling the scoliotic deformity. Although the illustrated embodiment of the implant 10 spans six vertebral levels, it should be understood that the implant 10 may be configured to span fewer or more vertebral levels.

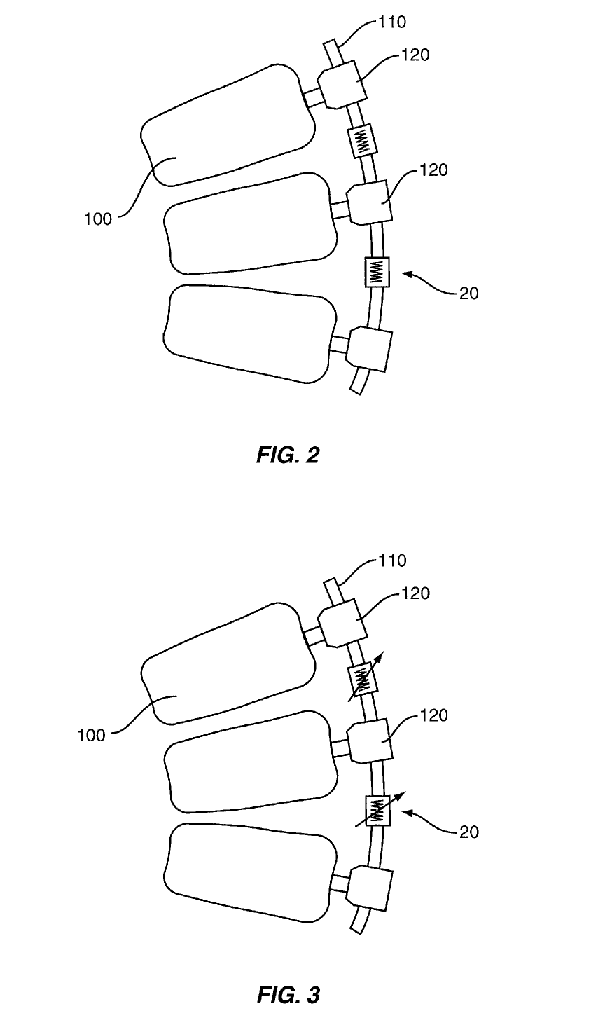

The implant 10 further includes one or more energy storing devices 20 attached to or forming a part of the tether 110. The energy storing devices 20 increase the range of extension between vertebral bodies. Further, the energy storing devices 20 tend to isolate distraction forces from the fasteners 120. FIG. 2 depicts a schematic representation of the energy storing devices as including a biasing device, such as a spring. In one embodiment, the energy storing device 20 may include a coil spring that is secured at opposite ends to the tether. In one embodiment, the energy storing device 20 includes a spring constant that is smaller than a corresponding spring constant of the tether 110. Consequently, when a tension force is applied to the combination tether 110 and energy storing device 20, a substantial majority of the resulting extension is attributable to the energy storing device 20. As the energy storing device 20 is extended, the extension force is converted to potential energy that is stored within the device 20. As the extension force is removed, the potential energy is converted to restoring the original, natural height of the energy storing device 20. Thus, the energy storing device 20 is substantially elastically deformable. In one embodiment, the energy storing device 20 may be at least partially plastically or permanently deformable. Notably, the energy storing device 20 need not include a fixed spring rate. FIG. 3 illustrates an embodiment of an energy storing device 20 including a variable spring rate. The effect of variable spring rates with different embodiments are described in greater detail below.

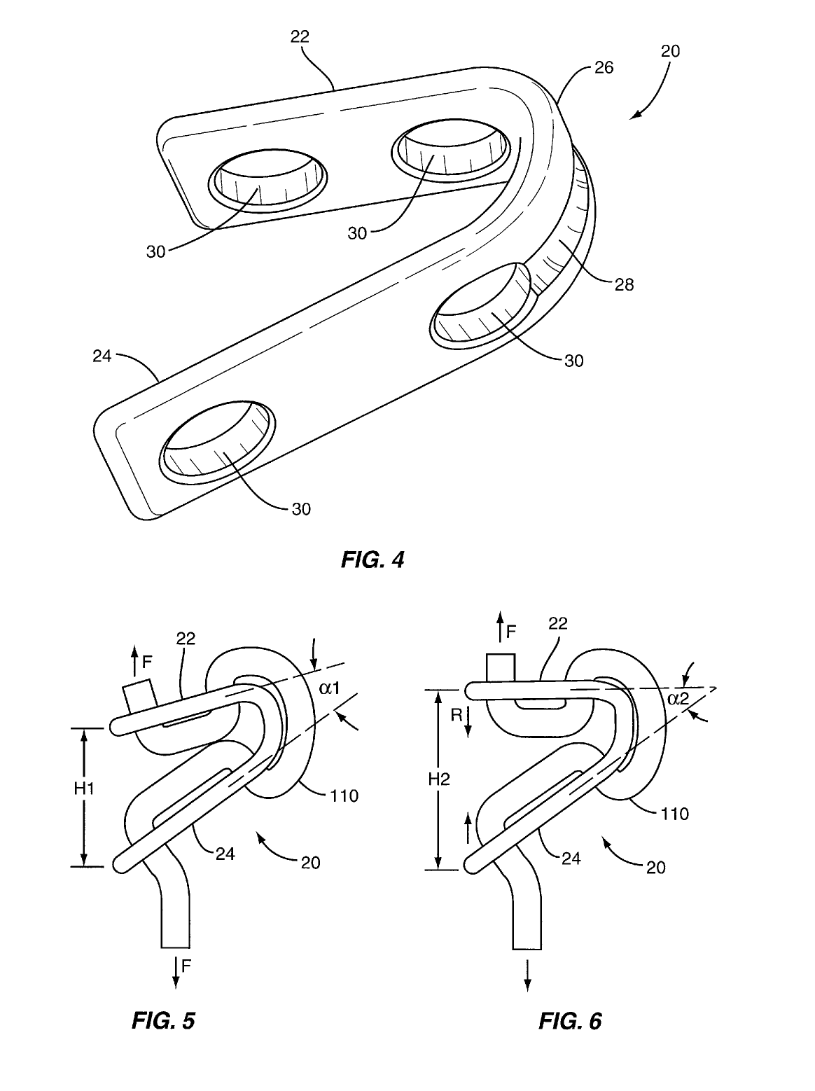

The energy storing device 20 may assume a variety of shapes or sizes. In addition to coil-type springs, the energy storing device 20 may assume a leaf spring shape as shown in the embodiment provided in FIGS. 4-6. Other designs, including embodiments described herein may include wire or cable configurations. In the embodiment illustrated in FIG. 4, the energy storing device 20 includes first 22 and second 24 arms extending from an intermediate bend 26. A groove 28 may be disposed along the convex side of the bend 26 and sized to receive a tether 110. Further, each of the first and second arms 22, 24 include apertures 30 that are sized to accept the tether 110. In the embodiment shown, the apertures 30 are substantially circular. The apertures 30 may include other shapes, such as oval, elliptical, teardrop, square, triangular, or other shapes that would occur to one skilled in the art.

FIG. 5 illustrates a lateral view of the energy storing device 20 with a tether threaded through the various apertures 30. The first and second arms 22, 24 include a generally acute angle α1 therebetween. In a neutral first state shown in FIG. 5, the ends of the arms 22, 24 opposite the bend 26 are spaced apart a distance H1. The tether 110 is threaded through the apertures 30 in the arms 22, 24 in such as manner that as an extension force F is applied to the tether 110, the first and second arms 22, 24 separate to a greater distance H2 as shown in FIG. 6. The potential energy stored in the energy storing device 20 tends to restore the first and second arms 22, 24 to the original distance H1 by a restoring force R. In the extended state shown in FIG. 6, the arms 22, 24 are separated by a greater angle α2, which happens to remain acute in the present embodiment.

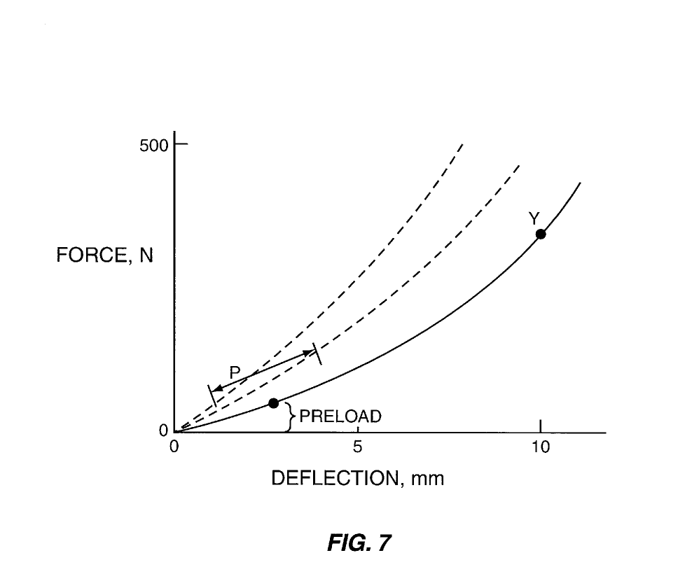

As discussed above, the energy storing device 20 may include a variable spring rate as the amount of deflection increases. FIG. 7 shows a representative force-deflection relationship for the energy storing device illustrated in FIGS. 4-6. In one embodiment, these exemplary numbers are provided by a leaf spring arrangement constructed from a super-elastic nickel-titanium alloy. In one embodiment, the energy storing device is constructed from a spring steel. Other biocompatible materials, including non-metals such as PEEK or UHMWPE may be used to construct the energy storing device 20. In one illustrative embodiment, the arms 22, 24 and bend 26 include a substantially similar cross section including a width of about 6-7 mm and a thickness of 1-2 mm. Those skilled in the art will comprehend that the energy storing device 20 may exhibit a variety of stiffnesses that are predictable based upon the elastic modulus of the material and the shape of the energy storing device.

FIG. 7 identifies two points of interest: P and Y. Point P represents a preload that is clinically applied to the tether 110 with the energy storing device 20. The tension is applied so that small extensions translate almost immediately to deflecting the energy storing device 20, which therefore operates as a buffer isolating shock forces from the fasteners 120 and vertebrae 100. The preload point P may be set at a non-zero force value. In one embodiment, the preload is set at a range between about 20N and 50N, though smaller or larger preloads are permissible. Point Y represents a failure point at the interface between the fasteners 120 and vertebrae 100. As shown, displacements below this failure point Y should produce an increasing return force in the energy storing device 20 that tends to return the tether 110 and vertebrae 100 a more neutral position. FIG. 7 includes one solid curve and two dashed curves to indicated that the energy storing device 20 may include different spring rates, which may be necessary for different patients, different regions of the spine, or different amounts of spinal correction/stabilization.

Within this range of desirable tension values, the energy storing device 20 is able provide a buffer against shock to the anchor locations at which the fasteners 120 engage the vertebral bodies 100. In use, the energy storage devices are able to convert extension forces that would otherwise cause damage to the vertebral bodies at the fasteners 120. It is generally understood that fasteners 120 such as bone screws tend to plow (i.e., enlarge the bone aperture in contact with the fastener threads) at a range between about 300 and 500 N. With tether devices, shock may occur due to sudden overextension of the tether, where the extension forces are suddenly translated to the anchor locations. Accordingly, the energy storing device 20 should be capable of preventing shock to the anchor locations between the fasteners 120 and vertebral bodies. In one implementation, the energy storing device 20 should limit shock to the anchor locations to a range between about 100-200 N and certainly below the 300-500 N failure range.

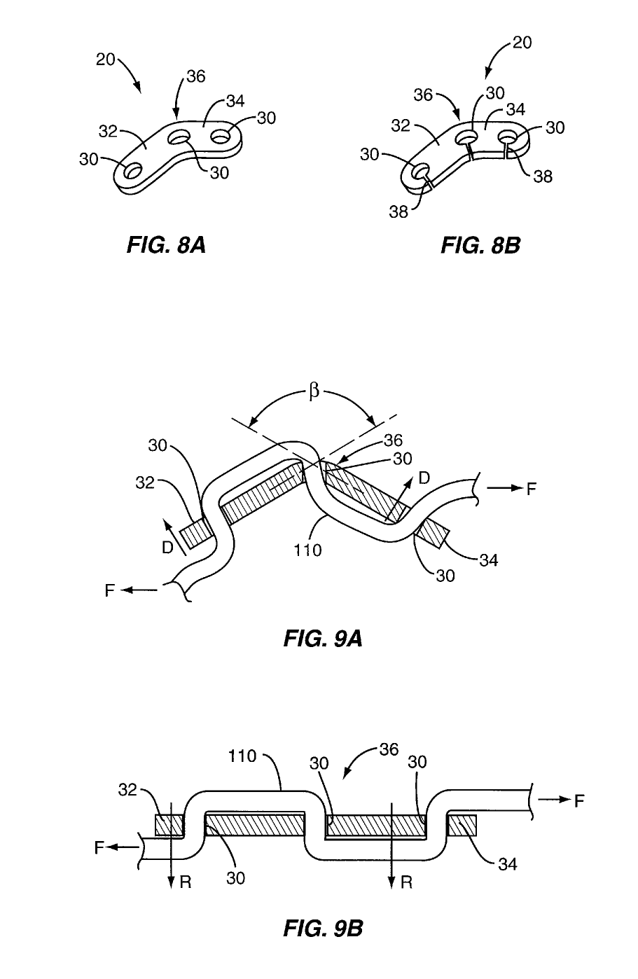

FIGS. 8A and 8B illustrate other embodiments of an energy storing device 20. These devices shown in FIGS. 8A and 8B are similar to each other in that each generally comprises an elongated body with first 32 and second 34 arms extending from an intermediate bend 36. Further, each energy storing device 20 includes a plurality of apertures 30 through which a tether 110 may pass. In the embodiment shown in FIG. 8B, the apertures 30 include an associated slot 38 that permits attachment of the energy storing device 20 to a tether 110 after the tether 110 is secured to fasteners. Specifically, the energy storing device 20 may be attached to a tether 110 by spreading apart the slots 38 to allow the tether 110 to pass through the slot 38 and into the aperture 30. In contrast, the embodiment shown in FIG. 8A requires that the tether 110 be threaded through the apertures 30 prior to securing the tether 110 to a group of fasteners 120.

FIGS. 9A and 9B respectively illustrate a side section view of the energy storing device 20 from FIG. 8A or 8B in a neutral and extended state. In FIG. 9A, the energy storing device 20 includes an obtuse angle β between the arms 32, 34. An extension force F applied to the tether 110 tends to increase this angle β and may deflect (force D) the energy storing device 20 to a substantially flat extended state shown in FIG. 9B. In use, the energy storing device 20 may not actually achieve the flattened configuration. In the extended state, the potential energy in the storing device 20 tends to restore the first and second arms 32, 34 to the original, neutral shape by a restoring force R.

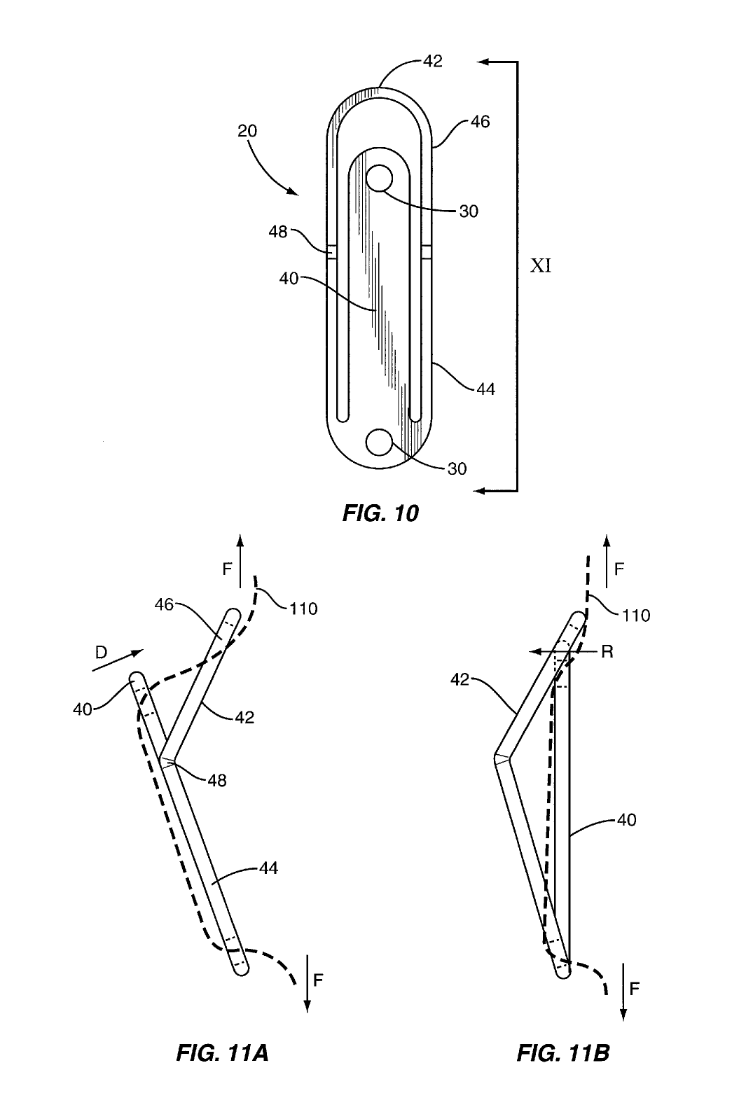

FIG. 10 shows an embodiment of an energy storing device 20 that includes a cantilevered central arm 40 that is deflectable relative to an arched frame 42. The central arm 40 includes apertures 30 through which a tether 110 may be threaded. FIGS. 11A and 11B respectively illustrate a side view of the energy storing device 20 from FIG. 10 in a neutral and extended state. In FIGS. 11A and 11B, a dashed line depicts the path along which the tether 110 passes. The arched frame 42 comprises an elongated body with first 44 and second 46 arms extending from an intermediate bend 48. As FIG. 11A shows, a tension force F applied to the tether tends to deflect the central arm 40 in the direction of deflection arrow D. Ultimately, the central arm 40 may deflect to the orientation shown in FIG. 11B. In this extended state, the potential energy in the storing device 20 tends to restore the central arm 40 to the original, neutral shape by a restoring force R.

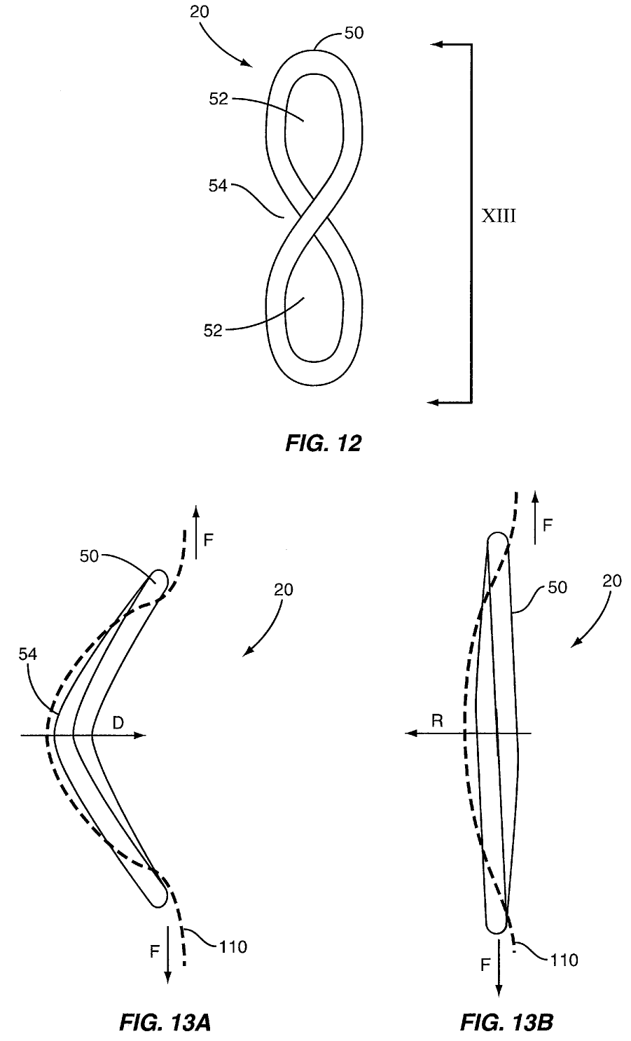

FIG. 12 shows an embodiment of an energy storing device 20 that includes an elastic wire 50 formed into a loop. In FIG. 12, the looped wire 50 forms a figure eight with the wire crossing at a central junction 54 and forming two tether apertures 52. FIGS. 13A and 13B further show that the looped wire 50 includes an arched configuration with a bend is formed at the central junction 54. Specifically, FIGS. 13A and 13B respectively illustrate a side view of the energy storing device 20 from FIG. 12 in a neutral and extended state. As FIG. 13A shows, a tension force F applied to the tether 110 tends to flatten the looped wire 50 and deflect the central junction 54 in the direction of deflection arrow D. Ultimately, the looped wire 50 may deflect to the orientation shown in FIG. 13B. In this extended state, the potential energy in the storing device 20 tends to restore the looped wire 50 to the original, neutral shape by a restoring force R.

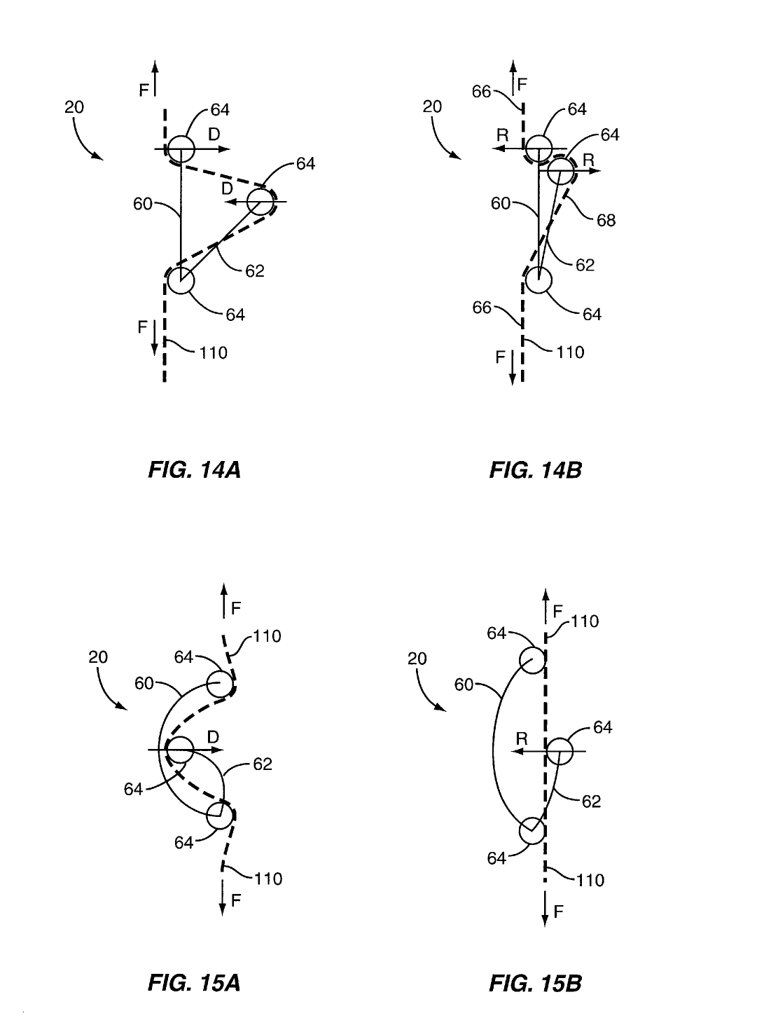

In embodiments described above, the tether 110 is generally looped through openings or apertures 30 in the energy storing device 20. As tension is applied to the tether 110, the energy storing device 20 deflects to an extended state, which also causes the tether 110 to assume a different shape at the interface with the energy storing device 20. For example, FIGS. 14A and 14B respectively illustrate a side view of an energy storing device 20 in a neutral and extended state. This exemplary energy storing device 20 may include wire arms 60, 62 and guides 64 about which the tether 110 is routed. The guides 64 may include rollers, bumpers, or other features sized to engage the tether 110. As with embodiments described above, a tension force F applied to the tether 110 tends to deflect the arms 60, 62 in the direction of deflection arrow D. Ultimately, the arms 60, 62 may deflect to the orientation shown in FIG. 14B. In this extended state, the potential energy in the storing device 20 tends to restore the looped wire 50 to the original, neutral shape by a restoring force R. Notably, in this embodiment, the tether 110 does not assume a substantially linear shape in either the neutral or extended states of FIG. 14A or 14B. The tether 110 approaches a linear shape in FIG. 14B, but a central region 68 of the tether 110 remains “kinked” or offset relative to the ends 66 of the tether 110 in vicinity of the energy storing device 20.

By contrast, the embodiment of an energy storing device 20 shown in FIGS. 15A and 15B includes a configuration similar to the embodiment shown in FIGS. 14A and 14B. That is, the energy storing device 20 in FIGS. 15A and 15B include arms 60, 62 and guides 64 about which the tether 110 is routed. However, in the embodiment in FIGS. 15A-B, the energy storing device 20 is configured to allow the tether 110 to substantially straighten (see FIG. 15B) when extended. As a consequence of this characteristic, the energy storing device 20 may slide relatively easily along the tether 110. Thus, a surgeon may roughly locate the energy storing device 20 at a desired location along the tether 110. Then, after inserting the tether 110 and device 20, the surgeon may fine tune the location of the device 20 along the tether by extending the device 20 while simultaneously adjusting the position of the device 20 relative to the tether 110. This should not suggest that other embodiments described herein are not adjustable in the same manner. However, the straightened shape of the tether 110 as shown in FIG. 15B should facilitate adjustment of the device 20 relative to the tether 110.

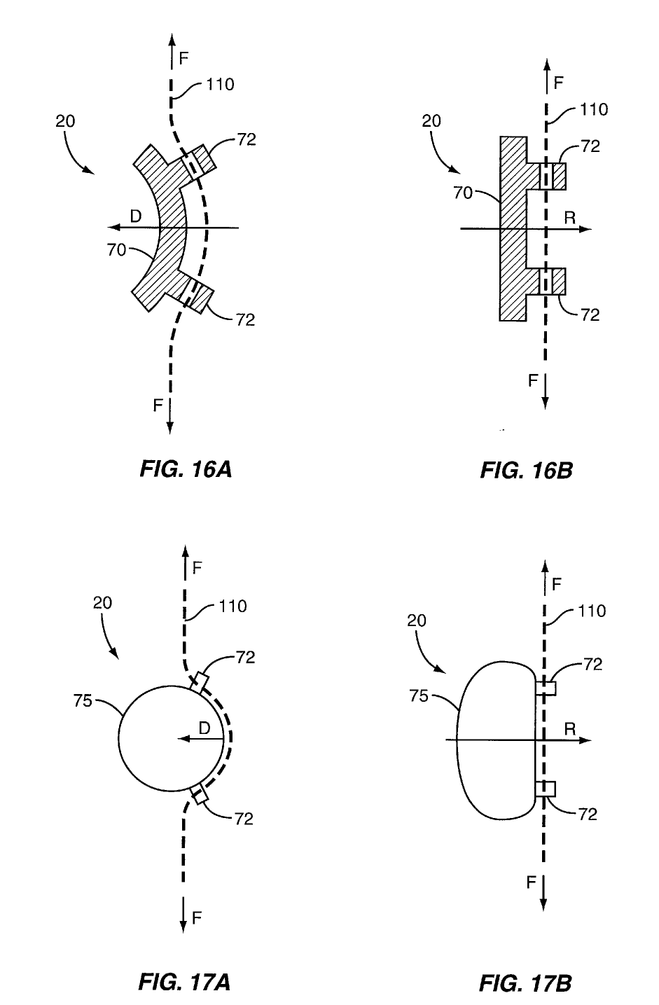

Embodiments of an energy storing device 20 shown in FIGS. 16A-B and 17A-B also include features that allow the tether 110 to assume a substantially straightened shape when the tether 110 is extended (see FIGS. 16B, 17B). In the embodiment shown in FIGS. 16A-B, the energy storing device 20 includes arched base 70 and two eyelets 72 extending outward from the convex side of the base 70. A tension force F applied to the tether 110 tends to deflect the arched base 70 in the direction of deflection arrow D. This deflection tends to straighten the arched base 70 as shown in FIG. 16B. Further, the eyelets 72 move towards a parallel orientation relative to each other. In this extended state, the potential energy in the storing device 20 tends to restore the arched base 70 to the original, neutral shape by a restoring force R. Notably, in this embodiment, the tether 110 assumes a substantially linear shape in the extended state of FIG. 16B.

In the embodiment shown in FIGS. 17A-B, the energy storing device 20 includes a compressible body 75 and two eyelets 72 extending outward from the body 75. The body 75 may include a spherical or cylindrical shape. Compressibility may be attributable to the body material or may be produced by a hollow body or an air or liquid filled body. A tension force F applied to the tether 110 tends to locally deflect the body 75 at the point of contact between the body 75 and tether 110 and in the direction of deflection arrow D. Further, the eyelets 72 move towards a parallel orientation relative to each other. In this extended state, the potential energy in the storing device 20 tends to restore the body 75 to the original, neutral shape by a restoring force R. Again, in this embodiment, the tether 110 assumes a substantially linear shape in the extended state of FIG. 17B.

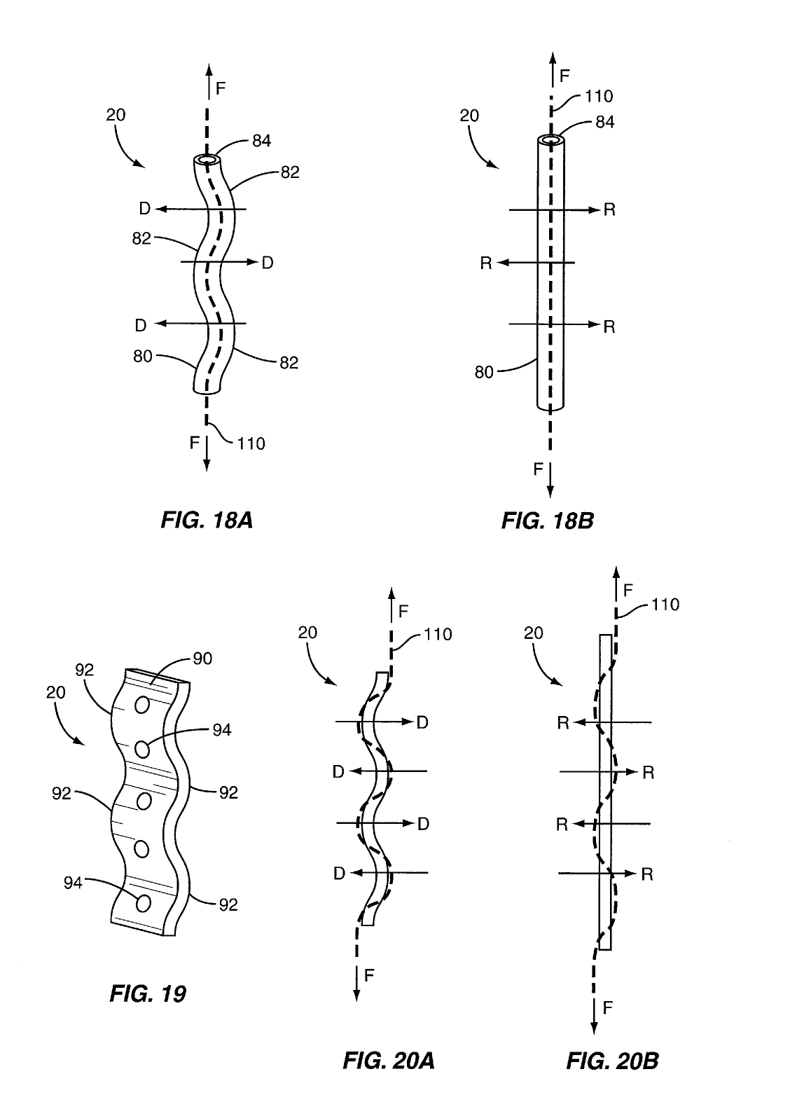

In the embodiment shown in FIGS. 18A-B, the energy storing device 20 includes an elongated tubular body 80 with laterally deviating bends 82. The tubular body 80 includes a hollow construction with an interior cavity 84 sized to allow a tether 110 to pass. A tension force F applied to the tether 110 tends to locally deflect the bends 82 at the point of contact between the tubular body 80 and tether 110 in the direction of deflection arrows D. That is, the tension force F tends to cause the tubular body 80 to straighten to the extended state shown in FIG. 18B. In this extended state, the potential energy in the storing device 20 tends to restore the tubular body 80 to the original, neutral shape by a series of local restoring forces R. In this embodiment, the tether 110 assumes a substantially linear shape in the extended state of FIG. 18B.

In the embodiment shown in FIGS. 19 and 20A-B, the energy storing device 20 includes a corrugated plate 90 with laterally deviating bends 92. The corrugated plate 90 includes a plurality of apertures 94 sized to allow a tether 110 to pass. That is, a tether 110 may be threaded through the apertures 94 along a path suggested by the dashed line in FIGS. 20A-B. When threaded in this manner, a tension force F applied to the tether 110 tends to locally deflect the bends 92 at the point of contact between the corrugated plate 90 and tether 110 in the direction of deflection arrows D. Consequently, the tension force F tends to cause the corrugated plate 90 to straighten to the extended state shown in FIG. 20B. In this extended state, the potential energy in the storing device 20 tends to restore the corrugated plate 90 to the original, neutral shape by a series of local restoring forces R.

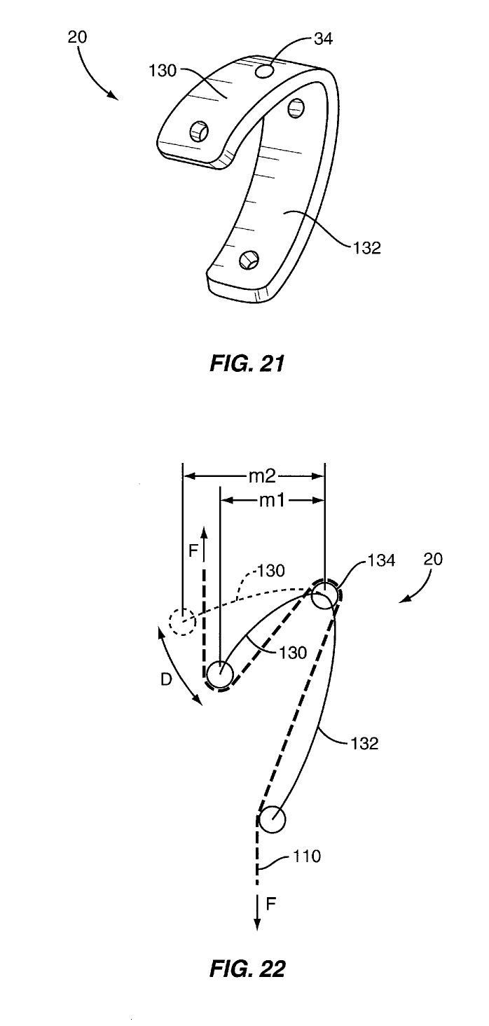

FIG. 21 illustrates an embodiment of an energy storing device 20 similar to the embodiment shown in FIG. 4. For example, the energy storing device 20 includes apertures 34 through which the tether 110 may be threaded. Further, the energy storing device 20 includes a first arm 130 and a second arm 132 that separate from each other when an extension force is applied to the tether 110. However, the present embodiment includes a slowly varying initial spring rate. A difference in this particular embodiment relates to the direction in which the first arm 130 separates from the second arm 132. To illustrate this characteristic, FIG. 22 shows a simplified schematic showing the relative positions of the arms 130, 132. As with other Figures, the path followed by tether 110 through the energy storing device 20 is shown as a dashed line. As the tether 110 is extended by the extension force F, the first arm 130 deflects in the direction indicated by arrow D. The dashed line representation of the first arm 130 shows the deflection of the first arm 130 relative to its original position.

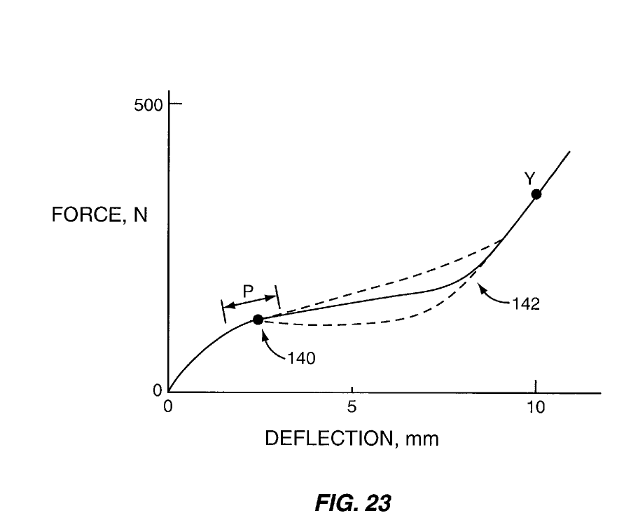

Each of the neutral and extended positions for the first arm 130 includes an associated moment arm M1, M2 that is the distance between the location at which the extension force F is applied and the center of rotation of the first arm 130. Notably, the moment arm M2 associated with the extended position is larger than the moment arm M1 of the neutral position. Thus, the moment arm increases, at least initially, under the influence of the extension force F. As a consequence of this increasing moment arm, the extension force F required to deflect the energy storing device 20 remains relatively static as compared to previously described embodiments. This force-deflection relationship is illustrated in FIG. 23.

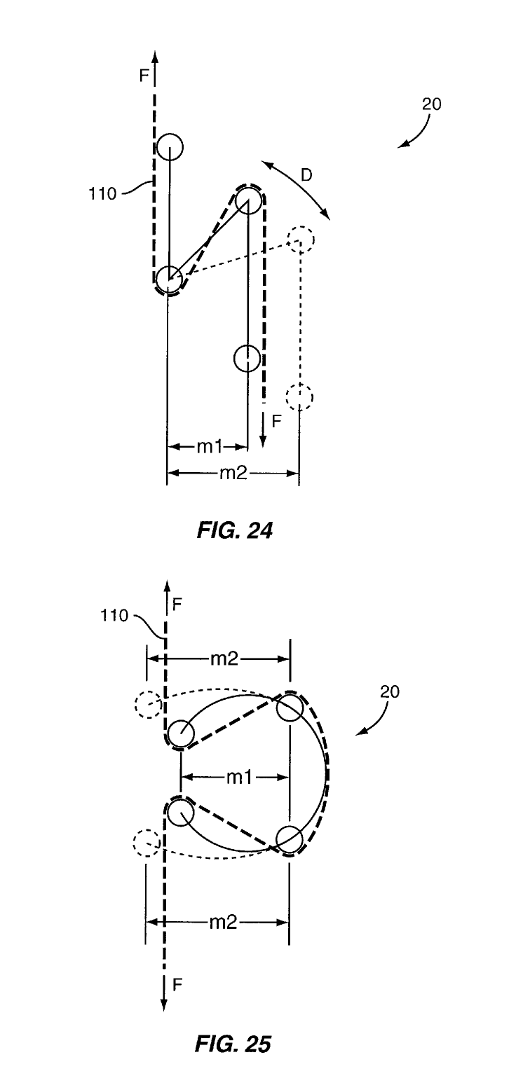

In contrast with the force-deflection relationship shown in FIG. 7, the graph in FIG. 23 includes two inflection points 140, 142. These inflection points generally indicate a change in the curvature of the spring rate curve (solid line). The first inflection point 140 indicates the point at which the curve begins to increase at a lower rate (due to the increasing moment arm). Beyond the first inflection point 140, the curve may be flat, rise slightly, or fall slightly (see dashed line representations) depending on the precise design of the energy storing device 20. In general, it may be desirable to include a flattened force-deflection curve to maintain a constant resistance provided by the energy storing device 20 over a normal range of motion. Then, as the tether 110 and energy storing device 20 become over-extended, the force-deflection curve may begin to sharply increase at the second inflection point 142. As with other embodiments, the energy storing device 20 may be pre-loaded to a desired force range. In one embodiment, this pre-load is between about 20N and 50N. In one embodiment, the pre-load may be set at or near the first inflection point for the energy storing device 20. Other energy storing device 20 shapes may achieve a relatively flat force-deflection curve. For example, FIGS. 24 and 25 respectively depict a Z-shaped energy storing device 20 and a C-shaped energy storing device 20. In each embodiment, the extension force F applied to the tether 110 initially causes an associated moment arm in the energy storing device 20 to increase. Consequently, the extension force F may be controlled to remain substantially flat (or slightly increase/decrease) during expected extension ranges. It should be noted that other shapes may achieve the same effect besides those described herein. For instance, an S-shaped energy storing device may achieve the same controlled force-deflection relationship.

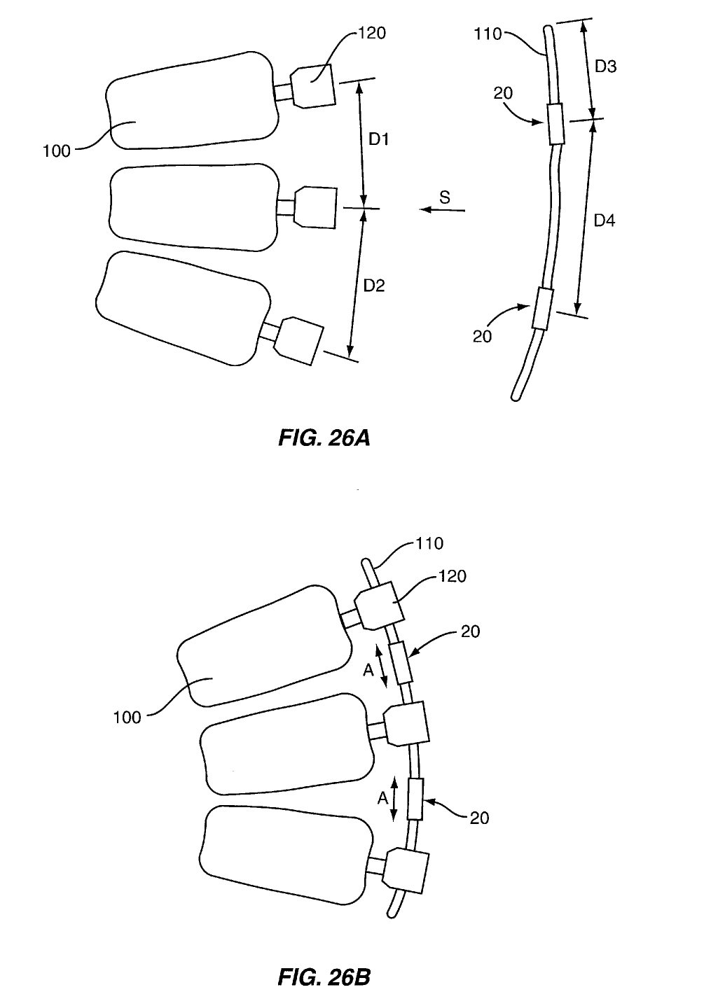

A surgeon may elect one of several different approaches to insert an energy storing device 20 and tether 110 into a patient. In one exemplary approach illustrated in FIGS. 26A-B, a surgeon may initially insert a suitable fastener 120 into select vertebral bodies to which correction is desired. Then, exterior to the patient, one or more energy storing devices 20 may be positioned onto a tether 110 in approximate positions as shown in FIG. 26A. The surgeon may loosely determine an approximate position for the energy storing devices 20 based upon measurable distances D1, D2 between the implanted fasteners 120. For example, the position of a first energy storing device 20 may be related to distance D1 while the position of a second energy storing device 20 may be related to second distance D2. The dimension D3 in FIG. 26A may be about half of distance D1 plus some excess to guarantee engagement of the tether 110 with the uppermost fastener 120 as shown in FIG. 26B. Similarly, dimension D4 may be equal to distance D1 or distance D2 or an average of D1 and D2. A similar approach may be followed for additional vertebral segments.

Once the energy storing devices 20 are loosely positioned, the tether 110, along with the attached device 20 may be inserted towards the fasteners 120 in the direction of arrow S. Once the tether 110 is at least partially secured to the plurality of fasteners 120, the attached energy storing devices 20 may be finely adjusted in the directions of arrows A. Then, a desired tension preload may be applied to the energy storing devices. This may be accomplished by applying an extension force to the tether 110 while securing the tether 110 to the fasteners 120.

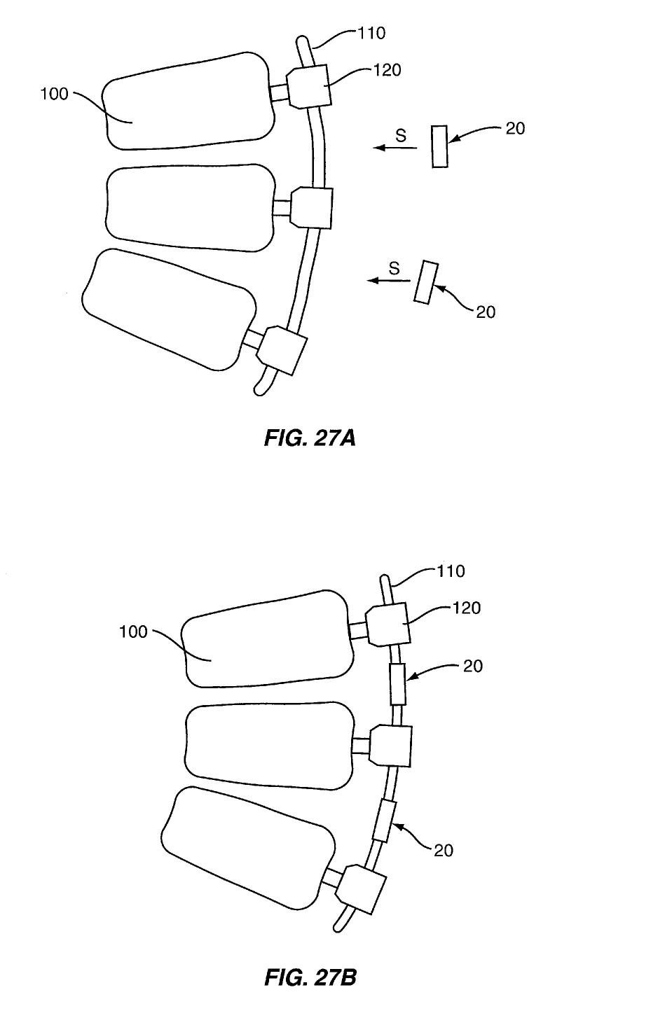

In another approach to inserting an energy storing device 20 and tether 110 into a patient, a surgeon may elect to attach the energy storing devices 20 to a previously implanted tether 110. FIGS. 27A-B broadly illustrate this process. The tether 110 may be implanted as an earlier step in a single operation or may be implanted in a prior operation. Thus, the energy storing devices 20 may be implanted as part of a revision surgery. Regardless, the energy storing devices 20 are inserted towards the implanted tether 110 generally in the direction of the arrows labeled S. A single energy storing device 20 is then attached to the tether 110 between pairs of adjacent fasteners 120 as shown in FIG. 27B.

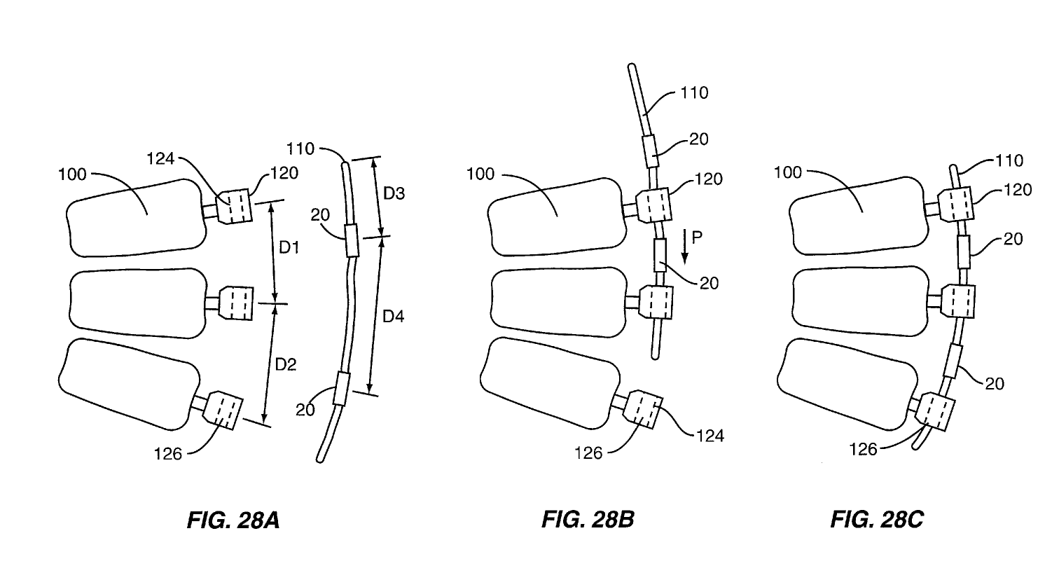

In another approach to inserting an energy storing device 20 and tether 110 into a patient, a surgeon may thread the tether 110 and attached energy storing devices 20 to previously implanted fasteners 120. Certain fasteners 120 may include a size and shape that requires lateral insertion of the tether 110 through the fastener 120. For example, the fastener 120 may include a head 124 with a laterally extending aperture 126. This particular approach may be used in open surgeries, but may find particular applicability in a percutaneous procedure, such as that illustrated in U.S. Pat. No. 6,899,713 to Shaolian et al., the relevant portions of which are incorporated by reference herein. Initially, the one or more energy storing devices 20 may be positioned onto a tether 110 in approximate positions as shown in FIG. 28A. The surgeon may loosely determine an approximate position for the energy storing devices 20 based upon measurable distances D1, D2 between the implanted fasteners 120 as described above. The tether 110, along with the attached device 20 may be threaded through the fasteners 120 in the direction of arrow P as shown in FIG. 28B. Clearly, this particular approach contemplates an energy storing device 20 that is sized to pass through the heads 124 of the implanted fasteners 120. Once the tether 110 is inserted to the position shown in FIG. 28C, the tether 110 may be secured to the plurality of fasteners 120.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. For instance, while the various figures and embodiments provided herein have described a single implant 10 and tether 110 attached to vertebral bodies 100, multiple implants 10 may be secured to the spine to further stabilize the spine. The present embodiments are, therefore, to be considered in all respects as illustrative and not restrictive, and all changes coming within the meaning and equivalency range of the appended claims are intended to be embraced therein.

What is claimed is:

1. An implant for attachment between vertebral members in a patient, the implant comprising:
a flexible tether including a length sufficient for attachment to at least two vertebral members; and
an energy storing device including an aperture sized to allow the tether to pass, the tether extending through the energy storing device with a first portion of the tether extending on a first side of the energy storing device and a second portion of the tether extending on an opposite second side of the energy storing device,
the energy storing device including a first neutral shape and assuming a second extended shape when an extension force is applied to extend the first and second portions of the tether away from the energy storing device.

2. The implant of claim 1 wherein the energy storing device comprises a spring.

3. The implant of claim 1 wherein the energy storing device comprises a leaf spring.

4. The implant of claim 1 wherein the energy storing device comprises a looped wire, the apertures formed by loops in the wire.

5. The implant of claim 1 wherein the energy storing device returns to the neutral shape after an extension force below a predetermined threshold is applied to extend the first and second portions of the tether away from the energy storing device and subsequently removed.

6. The implant of claim 5 wherein the predetermined threshold is between about 100 and about 200 Newtons.

7. The implant of claim 1 wherein the energy storing device comprises a tubular structure with the aperture extending through the tubular structure.

8. The implant of claim 1 wherein the energy storing device includes an increasing moment arm as the extension force is applied to extend the first and second portions of the tether away from the energy storing device to move the energy storing device from the first neutral shape to the second extended shape.

9. An implant for attachment between vertebral members in a patient, the implant comprising:
a first fastener secured to a first vertebral member;
a second fastener secured to a second vertebral member;
a flexible tether including a first portion secured to the first fastener and a second portion secured to the second fastener; and
an energy storing device coupled to the tether, the energy storing device including a first neutral shape and assuming a second extended shape when an extension force is applied to extend the first and second portions of the tether away from the energy storing device.

10. The implant of claim 9 wherein the energy storing device is coupled between the first and second portions of the tether.

11. The implant of claim 10 wherein the energy storing device is tensioned between the first and second portion of the tether.

12. The implant of claim 11 wherein the energy storing device is tensioned to between about 20 N and about 50N between the first and second portion of the tether.

13. The implant of claim 9 wherein the energy storing device comprises a spring.

14. The implant of claim 9 wherein the energy storing device comprises a leaf spring.

15. The implant of claim 9 wherein the energy storing device comprises a looped wire, the apertures formed by loops in the wire.

16. The implant of claim 9 wherein the energy storing device returns to the neutral shape after an extension force below a predetermined threshold is applied to extend the first and second portions of the tether away from the energy storing device and subsequently removed.

17. The implant of claim 16 wherein the predetermined threshold is between about 100 and about 200 Newtons.

18. The implant of claim 9 wherein the energy storing device comprises a tubular structure with the aperture extending through the tubular structure.

19. The implant of claim 9 wherein the energy storing device includes an increasing moment arm as the extension force is applied to extend the first and second portions of the tether away from the energy storing device to move the energy storing device from the first neutral shape to the second extended shape.

20. An implant for attachment between vertebral members in a patient, the implant comprising an energy storing member attachable to a vertebral tether, the energy storing member including a first neutral height in the absence of an externally applied force, and including a second extended height under the influence of an extension force below a predetermined threshold, the energy storing device returning to the neutral shape after the extension force below the predetermined threshold is removed.

21. The implant of claim 20 wherein the predetermined threshold is about 200N.

22. A method of stabilizing a spine using an implant that is attached between vertebral members in a patient, the method comprising:
securing a first fastener to a first vertebral member;
securing a second fastener to a second vertebral member;
securing a tether to the first and second fasteners;
coupling an energy storing device to the tether, the energy storing device including a first neutral shape in the absence of an external force applied to the tether and assuming a second extended shape when an extension force is applied to extend the tether away from the energy storing device; and
preloading the energy storing device to apply a predetermined tension on the tether.

23. The method of claim 22 wherein the step of preloading the energy storing device occurs by securing the tether to the first and second fasteners.

24. The method of claim 22 wherein the step of securing the tether to the first and second fasteners comprises threading the tether and energy storing device from a lateral direction through at least one of the first and second fasteners.

25. The method of claim 22 wherein the step of preloading the energy storing device to apply a predetermined tension on the tether further comprises preloading the energy storing device to a range between about 20N and about 50N.

26. The method of claim 22 wherein as the energy storing device assumes a second extended shape when an extension force is applied to extend the tether away from the energy storing device, the energy storing device exhibits an increasing moment arm.

27. The method of claim 22 wherein the step of coupling the energy storing device to the tether further comprises threading the tether through one or more apertures in the energy storing device.

28. The method of claim 22 wherein the step of coupling the energy storing device to the tether further comprises threading the tether through loops formed in an energy storing device formed from a coiled wire.

29. The method of claim 22 wherein the step of coupling the energy storing device to the tether occurs before the step of securing the tether to the first and second fasteners.

30. The method of claim 22 wherein the step of coupling the energy storing device to the tether occurs after the step of securing the tether to the first and second fasteners.

31. The method of claim 22 further comprising measuring a first distance between the first and second fasteners after they are respectively secured to the first and second vertebral bodies and coupling the energy storing device to the tether at a location that is associated with the first distance.
